(19) United States
(12) Patent Application Publication
(10) Pub. No.: US 2020/0284778 A1
(43) Pub. Date: Sep. 10, 2020

(54) FUEL TESTER FOR CHARACTERIZATION OF THE SUSCEPTIBILITY TO THERMOACOUSTIC INSTABILITIES AND METHOD

(71) Applicants: KING ABDULLAH UNIVERSITY OF SCIENCE AND TECHNOLOGY, Thuwal (SA); TECHNISCHE UNIVERSITÄT BERLIN, Berlin (DE)

(72) Inventors: Thibault F. GUIBERTI, Thuwal (SA); Francesco DI SABATINO, Thuwal (SA); Jonas P. MOECK, Trondheim (NO); Wesley R. BOYETTE, Thuwal (SA); Deanna A. LACOSTE, Thuwal (SA); William L. ROBERTS, Thuwal (SA)

(21) Appl. No.: 16/759,628

(22) PCT Filed: Oct. 16, 2018
(86) PCT No.: PCT/IB2018/058021

§ 371 Date: Apr. 27, 2020

Related U.S. Application Data
(60) Provisional application No. 62/631,037, filed on Feb. 15, 2018.
(60) Provisional application No. 62/582,048, filed on Nov. 6, 2017.

(51) Int. Cl. G01N 33/28; F23R 3/20

(52) U.S. Cl. CPC G01N 33/2817; F23R 3/20

(57) ABSTRACT

A fuel testing device includes a combustion chamber having an optical access port; a visualization system that acquires images of a flame inside the combustion chamber, the images being acquired through the optical access port; and a vortex generator that perturbs a flow of a fuel inside the combustion chamber. The images are used to determine a propensity of the fuel to thermoacoustic instabilities, and the combustion chamber has a length less than 2 m.

### CROSS-REFERENCE TO RELATED APPLICATIONS

This application claims priority to U.S. Provisional Patent Application No. 62/582,048, filed on Nov. 6, 2017, entitled “FUEL TESTER FOR CHARACTERIZATION OF THE SUSCEPTIBILITY TO THERMOACOUSTIC INSTABILITIES,” and U.S. Provisional Patent Application No. 62/631,037, filed on Feb. 15, 2018, entitled “FUEL TESTER FOR CHARACTERIZATION OF THE SUSCEPTIBILITY TO THERMOACOUSTIC INSTABILITIES AND METHOD,” the disclosures of which are incorporated herein by reference in their entirety.

### BACKGROUND

### Technical Field

Embodiments of the subject matter disclosed herein generally relate to methods and devices for fuel testing, and more specifically, to methods and systems for determining the susceptibility to thermoacoustic instabilities of a gas turbine due to fuel changes.

### Discussion of the Background

When the fluctuations of the heat release rate of a flame couple with an acoustic mode of a combustion chamber, one refers to thermoacoustic coupling, thermoacoustic oscillations, or thermoacoustic instabilities. Thermoacoustic instabilities can lead to high-amplitude oscillations of the pressure, the flow field, and the flame, which can in turn increase noise and pollutant emissions, as well as decrease the efficiency of the combustion system, heat transfer to the combustor walls, and flashback or blowout. In severe cases, the thermoacoustic instabilities could lead to structural failure of the gas turbine. Avoiding the occurrence of thermoacoustic instabilities is a major challenge in the design of stationary gas turbines and aero-engines.

Combustion instabilities arise when the unsteady combustion process couples with the acoustic modes of the combustor. This phenomenon is therefore dependent on the geometry of the combustor, the fuel characteristics, and the settings of the gas turbine. To understand and predict thermoacoustic instabilities, it is needed to understand the response of the flame to acoustic perturbations. This response depends on many parameters, among them, the composition of the unburned mixture, the mean flame shape, the flow field, and the operating temperature and pressure.

A common approach to quantify the response of the flame to acoustic perturbations is through the formalism of the flame transfer functions (FTFs). These transfer functions are deduced from the systematic analysis of the heat release rate (HRR) fluctuations of a flame subjected to a controlled acoustic forcing, with forcing frequencies ranging from a few hertz to typically a few hundred hertz. This approach can lead to the understanding of the forcing mechanisms, and FTFs are considered as a tool in predicting the flame sensitivity to thermoacoustic instabilities.

Unfortunately, as the flame dynamics depend on many parameters, results obtained at given conditions are difficult to extrapolate. Thus, full-scale tests are currently the standard way of characterizing any new design. This means that the various fuels are tested on the actual gas turbines. This process is undesirable because the tests are time consuming and very particular to the gas turbine that is being tested.

On the other hand, the life cycle of gas turbines and aero-engines is usually more than 20 years. During this long period of time, it is more than likely that the engines will have to face changes in the fuel composition. For aero-engines, due to safety issues and associated severe regulations, fuel changes are minimal. However, for gas turbines used in ground power plants, the expected fuel composition changes can be significant. Each time the fuel composition is changed (for example through changes of supplier, seasonal fluctuations, or transition to next generation bio-fuels and hydrogen-enriched fuels), the propensity of the combustor to exhibit thermoacoustic instabilities changes. Thus, new tests must be conducted in order to validate the new operating settings. These tests are very costly as they require a shutdown of the turbine, which reduces productivity.

Thus, there is a need for a new way of testing, that is cheaper and faster than the full-scale tests, to characterize any fuel or any changes in the fuel composition with respect to their propensity to produce flames susceptible to thermoacoustic instabilities. Such a new way would reduce the number of these full-scale tests, or even make them entirely redundant.

### SUMMARY

According to an embodiment, there is a fuel testing device that includes a combustion chamber having an optical access port, a visualization system that acquires images of a flame inside the combustion chamber, the images being acquired through the optical access port, and a vortex generator that perturbs a flow of a fuel inside the combustion chamber. The images are used to determine a propensity of the fuel to thermoacoustic instabilities, and the combustion chamber has a length less than 2 m.

According to another embodiment, there is a method for testing a new fuel for a gas turbine. The method includes a step of providing the new fuel to a fuel testing device that includes a combustion chamber, a step of applying a set of three parameters to the fuel testing device, wherein the set of three parameters are substantially the same as for the gas turbine, and wherein the set of three parameters are related to (i) the new fuel, (ii) an oxidizer, and (iii) a pressure inside the combustion chamber, a step of perturbing a flow of the new fuel and the oxidizer with a given acoustic frequency, a step of burning the perturbed flow of the new fuel and the oxidizer in the combustion chamber to generate a flame, and a step of comparing a parameter of the flame of the new fuel with a corresponding parameter of a flame of a reference fuel. Based on a result of the comparing step, the new fuel is determined to have more or less thermoacoustic instabilities than the reference fuel.

According to still another embodiment, there is a method for testing a new fuel for a gas turbine. The method includes providing the new fuel to a fuel testing device that includes a combustion chamber; applying a set of three parameters to the fuel testing device, where the set of three parameters are substantially the same as for the gas turbine, and wherein the set of three parameters are related to (i) the new fuel, (ii) an oxidizer, and (iii) a pressure; perturbing a flow of the new fuel and the oxidizer with a given acoustic frequency; burning the perturbed flow of the new fuel and the oxidizer in the combusting chamber to generate a flame; comparing a parameter of the flame of the new fuel with a corresponding parameter of a flame of a reference fuel; and estimating a propensity for thermoacoustic instabilities of the new fuel relative to the reference fuel based on a maximal size of a ball of the flame and a phase difference between (1) an acoustic perturbance propagating through the new fuel, and (2) a flame heat release fluctuation due to the given acoustic perturbation.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1 illustrates a schematic of a fuel testing device;

FIGS. 2A and 2B show an implementation of the fuel testing device;

FIGS. 3A and 3B show a method for using the fuel testing device and determining a propensity of a new fuel for thermoacoustic instabilities;

FIGS. 4A to 4E illustrate images of the flames inside the fuel testing device recorded with a visualization system;

FIG. 5 illustrates a phase difference between an acoustic disturbance propagating in the combustion chamber and a heat released by the burning fuel;

FIGS. 6A to 6F illustrate how the ball of a flame is selected;

FIG. 7 is a flow chart of a method for determining the propensity of a new fuel to thermoacoustic instabilities; and

FIG. 8 is a schematic illustration of an analysis unit.

### DETAILED DESCRIPTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to a gas turbine. However, the invention is not limited to this scenario, but it may be used for other devices that burn fuel together with an oxidizer for producing energy.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a fuel testing device is used to burn a new fuel to determine the propensity of the new fuel to thermoacoustic fluctuations. Various conditions (to be discussed later) are imposed on the fuel testing device in order to reproduce the thermoacoustic fluctuations that would appear in a real gas turbine. Note that the fuel testing device is much smaller than the gas turbine, e.g., between 10 to 100 times smaller. In one application, the fuel testing device is portable, i.e., it can be moved from one location to another location by one or more persons while a gas turbine is so large that one or more cranes are necessary for moving it. Further, the testing device uses a smaller amount of fuel for the testing and the testing time is substantially reduced. In this regard, while an actual gas turbine is required to run for hours if not days for testing a new fuel and its propensity to thermoacoustic fluctuations, the fuel testing device can achieve the same results in minutes.

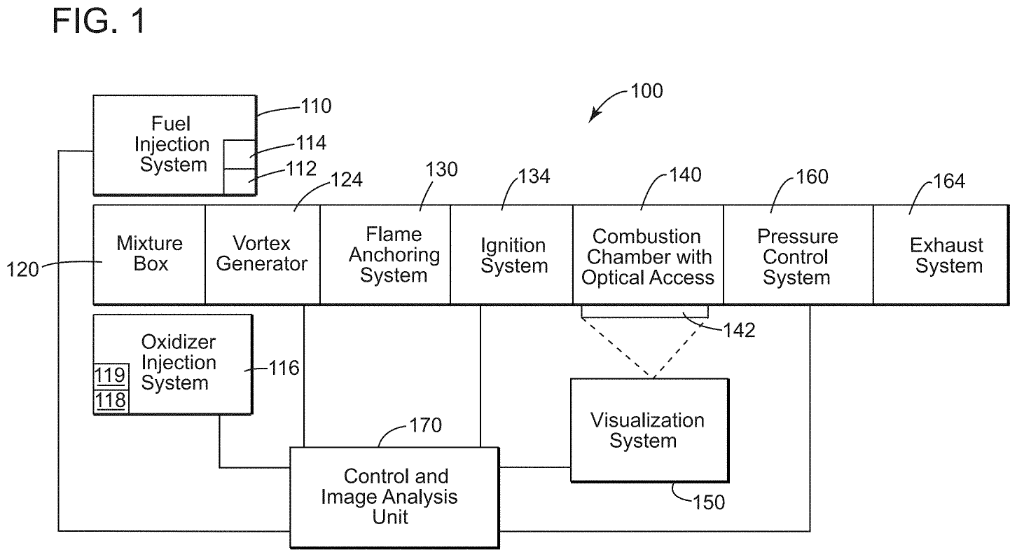

A fuel testing device 100 is schematically shown in FIG. 1. According to this embodiment, the fuel testing device 100 has a fuel injection system 110 and an oxidizer injection system 112 that feed the fuel and the oxidizer to a mixing box 120. The fuel injection system may include, for example, a mass flow controller 112 associated or not with a temperature control device 114 that controls a temperature of the fuel. The fuel provided by the fuel injection system is the new fuel that needs to be tested. The oxidizer injection system 116 may provide the oxidizer (e.g., air, oxygen) for burning the fuel. The oxidizer injection system may include, for example, one or more mass flow controllers 118, associated or not, with a temperature control device 119 for controlling a temperature of the oxidizer. The mixing box 120 can be any device or tubing allowing a mixture of the fuel and oxidizer (reactants) to flow through. The mixing box may, or may not, be thermalized.

The fuel testing device 100 further includes a vortex generator 124, which is a device that allows the generation of vortices of a controlled size and azimuthal vorticity in the combustion chamber. Various vortex generators may be used, for example, a cam shaft, a rotating valve, a piston, a loudspeaker, any device that would change the pressure and/or volume of an enclosure through which the reactants flow. In order to control the size of the vortex in the flame area, the vortex generator should be able to modulate the flow of reactants at a given frequency. This frequency should be in the range of 10 Hz to 1 kHz (these are the practical frequencies for a gas turbine), and the effective frequency is calculated based on the actual dimensions of the fuel testing device, the flame in the combustion chamber, and the average bulk velocity of the reactants, as discussed later.

The fuel testing device 100 may also include a flame anchoring system 130. After being perturbed by the vortex generator 124, the flow of reactants is injected into the combustion chamber 140 through a cylindrical injector featuring a typical dimension D. Dimension D is chosen in this embodiment to provide an injector cross-section of 20×20 square millimeters. Other values for this value may be used. Downstream the injection systems 110 and 116, the flame anchoring system 130 forces the flame to stabilize and burn inside the combustion chamber 140. The flame anchoring system 130 can be made of a swirler or a bluff body (i.e., an obstacle), or any other passive device that forces the flame to burn within the combustion chamber.

The fuel testing device 100 may further include an ignition system 134. The ignition system may be any known flame ignition system, for example, an electrical spark, a laser spark, a hydrogen torch, a hot surface, etc. In one embodiment, the ignition system 134 may be installed in the combustion chamber 140.

The fuel testing device 100 may further include the combustion chamber 140. The combustion chamber 140 needs to have an optical access port 142. After ignition of the reactants by the ignition system 134, the flame is stabilized in the combustion chamber 140. The optical access port 142 allows a visualization system 150 to take pictures (photography or movies) of the whole flame. The flame should be entirely located inside the combustion chamber and the combustion chamber needs to be big enough to avoid a direct interaction between the flame and the wall of the combustion chamber. This is so because it is desired to have the flame independent of the geometry of the combustion chamber and this happens only if the flame does not interact with the walls of the combustion chamber. In one application, the inner volume of the combustion chamber 140 for the fuel testing device 100 should be in the range of 10 cubic centimeters to 10 liters. Those skilled in the art would understand that the inner volume may take other values.

The combustion chamber 140 can be of any shape, for example, cylindrical, rectangular, cubic, etc. The temperature of the walls and of the optical access port (which may be a window) may, or may not, be actively controlled. In one application, the combustion chamber and the rest of the modules of the fuel testing device 100 should operate at pressures up to 50 bar.

To control the pressure inside the fuel testing device 100, a pressure control system 160 (e.g., a valve) may be used. The pressure control system 160 may be located at any point in the fuel testing device. The pressure control system's function is to maintain the static pressure in the combustion chamber 140, to any specified value between 1 and 50 bar.

After combustion of the reactants in the combusting chamber 140, the burnt gases are released to the atmosphere by an exhaust system 164. The exhaust system 164 can be made of pipes or of any system that is resistant to the hot exhaust gases.

The visualization system 150 images the flame in the combusting chamber 140 with a camera or equivalent means. These images would be used (as discussed later) to determine a maximum size of a flame ball. This camera can be a fast camera (with an acquisition rate of at least 1000 frames per second) or a regular camera that can be triggered and synchronized with the vortex generator 124. This camera can be a color or grayscale camera. Alternatively, an array of photodiodes can be used to record the temporal evolution of the light emission from the flame with a good spatial resolution (in the range of 10 micron to 10 mm). The temporal evolution of the flame shape and its size will be the quantities that will be used (discussed later) to characterize the fuel with respect to its propensity to produce thermoacoustic instabilities.

A control and image processing unit 170 may be added to the fuel testing device 100 for controlling the various components discussed above and/or processing the images acquired by the visualization system 150. However, this unit is not necessary for the fuel testing device because control and image processing can be performed by a trained operator. An advantage of implementing unit 170 is the simplification that results in the use of the fuel testing device and the decrease in the tests duration. In one application, computer routines needed to control the fuel testing device 100 and process the images recorded by the visualization system 150 may be coded and implemented via any programing language (e.g., C++, Fortran, or commercial software such as Matlab).

In one implementation, the unit 170 controls the fuel injection system 110, the oxidizer injection system 116, the vortex generator 124, the ignition system 134, the visualization system 150, and the pressure control system 160 and the synchronization with the visualization system 150. One goal of the control and image analysis unit 170 is to extract the dimension of the flame as well as the size of the flame ball as these parameters reflect the sensitivity of the flame to wrap around the vortex (flame/vortex roll-up).

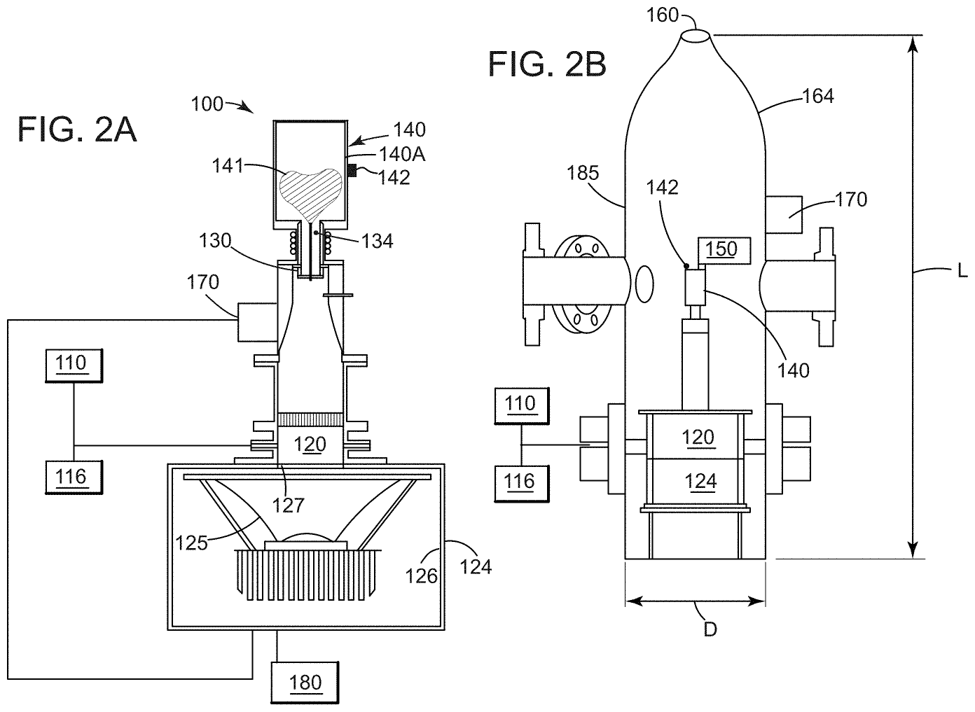

An actual implementation of the fuel testing device 100 is now discussed with regard to FIGS. 2A and 2B. FIG. 2A shows the fuel testing device 100 without the high pressure duct and the pressure control system while FIG. 2B shows these elements and the optical access port 142 of the combustion chamber 142. More specifically, FIG. 2A shows the fuel injection system 110 and the oxidizer injection system 116 mixing the fuel (for example, methane and propane) and oxidizer (e.g., air) outside the mixing box. The mixed reactants are then injected into the mixing box 120. The flow of the reactants may be controlled by mass flowmeters (e.g., Brooks SLA 58 series; other flowmeters may be used).

Mixing box 120 is shown in FIG. 2A being located on top of the vortex generator 124. In this embodiment, the vortex generator 124 includes a loudspeaker 125 placed inside an enclosure 126. Enclosure 126 has a top opening 127 that communicates with mixing box 120. By controlling a frequency and voltage being applied to the vortex generator 124, for example, with the control and image analysis unit 170, the flow of the reactants in the mixing box 120 may be perturbed as desired for studying the propensity of the new fuel to thermoacoustic instabilities. A power source 180 may provide the required voltage to the vortex generator 124 and to any other component in the fuel testing device 100 that requires electrical power.

The mixed reactants flow from the mixing box 120 (in this case a plenum having a length of about 120 mm; those skilled in the art would understand that this number is exemplary and not intended to limit the application of the invention) into the flame anchoring system 130, which in this embodiment is a radial swirler. The radial swirler quickly mixes up the reactants prior to ignition.

The mix of reactants is ignited by ignition system 134 (a laser spark in this embodiment) and burned inside the combustion chamber 140. FIG. 2A shows a flame 141 formed inside the combustion chamber 140. As discussed above, this flame should not touch the walls 140A of the combustion chamber 140. FIG. 2A shows the optical port 142 formed in the wall 140A of the combustion chamber 140. In one embodiment, the combustion chamber 140 may be made of quartz, in which case any part of the wall 140A may be used as the optical access port. In one embodiment, a length of the combustion chamber is about 100 mm and its inner diameter is about 70 mm. These dimensions are provided to offer the reader a feeling about the small size of the fuel testing device 100 in comparison to an actual gas turbine, which has a length in the order of meters to tens of meters.

The burnt gases are exhausted through the exhaust system 164, which is shown in FIG. 2B. The pressure control system 160 is shown in this figure being placed at the top of the exhaust system 164. Note that FIG. 2B shows a housing 185 in which the various components of the fuel testing device 100 are located. FIG. 2B also shows the optical port 142 formed also in the housing for permitting access of the visualization system 150 to the flame 141. Depending on the embodiment, the optical access port 142 is understood to be formed in a wall of the combustion chamber 140, if that wall is opaque, or a wall of the housing 185, or both.

In one embodiment, a length L of the entire fuel testing device 100 may be in the order of tens to hundreds of mm and a diameter of the device may be in the order of tens of mm. In one application, the length L is smaller than 2 m. These exemplary ranges suggest to one skilled in the art the small size of the fuel testing device relative to the actual gas turbine. However, those skilled in the art would understand that there are no restrictions in increasing or further decreasing the sizes of the fuel testing device 100.

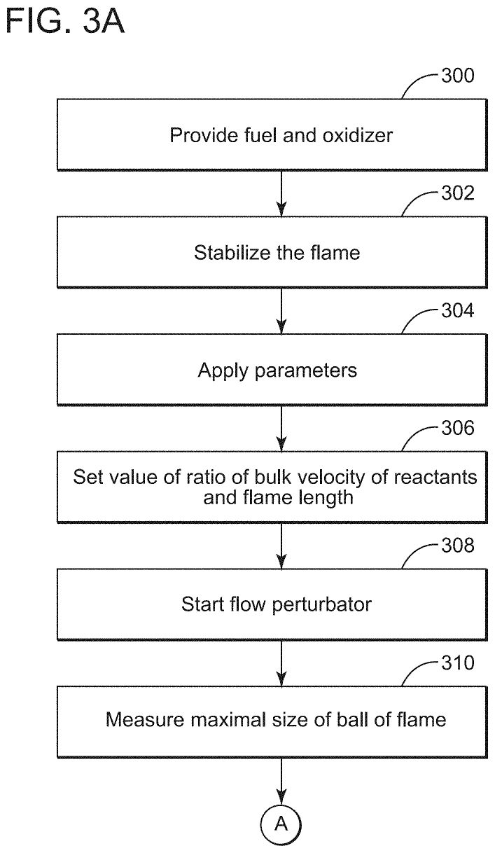

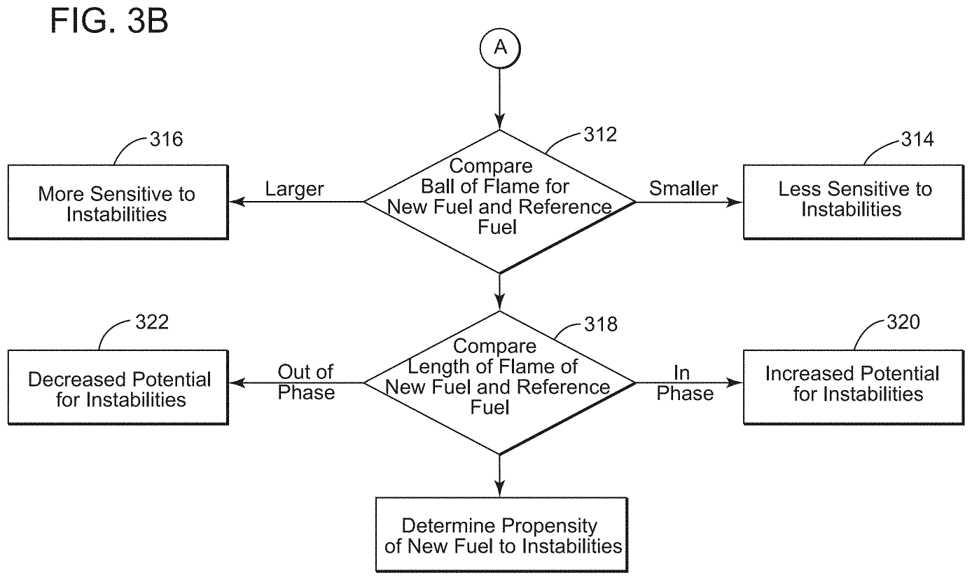

Having the benefit of the fuel testing device 100 discussed above, a method for testing the propensity to thermoacoustic instabilities of a given fuel is now discussed with regard to FIGS. 3A and 3B. In step 300, the fuel and the oxidizer are provided to the combustion chamber or the fuel testing device and the mixture is ignited to generate a flame. The fuel used in this method may be any fuel. In one application, the method to be discussed herein is run first with a traditional fuel (reference fuel) that has been used in a given gas turbine and then the method is repeated for a new fuel that is desired to replace the traditional fuel in that gas turbine.

In step 302, the flame is stabilized. The fuel testing device 100 is controlled/adjusted, for example, by the control and image analysis unit or by the operator of the device, such that the produced flame is stabilized in the combustion chamber 140. In this step, the vortex generator 124 is inactive, i.e., no perturbation is applied to the flow of reactants.

Three parameters associated with reactants and the pressure inside the fuel testing device need to be controlled to reproduce the thermoacoustic instabilities in an actual gas turbine. These three parameters are first defined and then discussed in the context of the fuel testing device.

The first parameter is the equivalence ratio, i.e., the fuel to oxidizer proportion. The second parameter is the type of oxidizer, the air. The third parameter is the pressure inside the fuel testing device. The inventors of this application have observed that if these three parameters for the actual gas turbine are the same for the fuel testing device, then the fuel testing device correctly and accurately describes the propensity to thermoacoustic instabilities of the new fuel to be used in the actual gas turbine.

This means that in step 304, the equivalence ratio, the oxidizer and the pressure inside a given gas turbine are received and the same parameters are applied to the fuel testing device 100. The term the “same” in this context means that corresponding parameters are considered to be equal if a variation of the actual parameters of the gas turbine and the fuel testing device is smaller than 20% of the actual value of the parameter for the gas turbine.

One skilled in the art would understand that the three parameters may be measured or set at the gas turbine and then the same parameters are adjusted at the fuel testing device, for example, by controlling the fuel injector system 110 and the oxidizer injection system 116 for the equivalence ratio parameter, the oxidizer injection system 116 for the oxidizer parameter, and the pressure control system 160 for the pressure parameter.

In step 306, the ratio between (1) the bulk velocity U of the reactants (which can be calculated from the dimensions of the fuel testing device and the average flow of the reactants) and (2) the flame size L is set to be equal to a given value, Str. This value is dictated by the operating frequency F of the vortex generator 124 via the formula: F=0.5U/L, where Str=0.5. Note that the ratio between the bulk velocity of the reactants and the flame size L should be the same for two different fuels that are tested in the fuel testing device 100, in order to have a meaningful comparison of the two fuels. However, this ratio is not required to be the same for a fuel that is tested in the fuel testing device and a fuel used in the actual gas turbine. In other words, the ratio used in step 306 serves to compare various fuels tested in the fuel testing device for determining which fuel would perform with least amount of the thermoacoustic instabilities in the actual gas turbine.

Returning to the formula F=0.5U/L, it can be rewritten as 0.5=(FL)/U. The frequency F is constant after the vortex generator 124 has been set up, the length L of the flame usually has a desired value, and thus, the bulk velocity U of the reactants needs to be adjusted to fulfill the formula noted above.

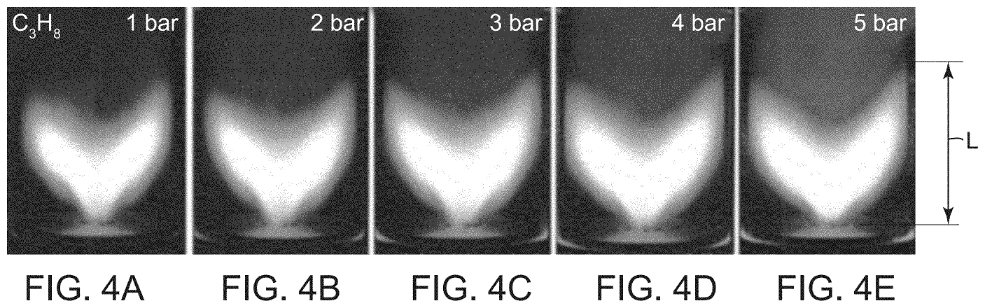

For calculating the flame size L, the visualization system 150 may be used. For example, as illustrated in FIGS. 4A-4E, the visualization system 150 records images of the flame (through the optical access port 142). Note that C₃H₄ is burnt for generating the flames shown in FIGS. 4A-4E at an increasing pressure (from 1 to 5 bar). The control and image analysis unit 170 may then analyze an image of the flame, for a given pressure, and calculate the length L of the flame. The ends of the flame are determined by considering where a luminosity is detected by the unit 170. Note that a flame is considered in the art to be associated with a given set of wavelengths and thus, the luminosity is associated only with those wavelengths. Where no luminosity is determined, it is assumed that there is no flame. FIGS. 4A to 4E also show how the length L of the flame is maintained substantially constant for varying pressures.

In step 308, the vortex generator 124 is started. The flame will then begin to move and change its shape due to the propagation of vortices in the combustion chamber. In step 310, the maximal size of the flame ball generated by the winding of the combustion front on the vortex (also called vortex roll-up) is measured. This step may be achieved with a fast camera (e.g., the visualization system 150) or, alternatively, by using a regular camera synchronized with the vortex generator 124 because the maximal size of the flame ball can appear at any phase of a forcing period.

To measure the flame ball, the following procedure may be used. First, for each experimental condition (fuel, equivalence ratio, pressure), the fuel testing device 100 is run for about 15 minutes to ensure that the burner has reached thermal equilibrium. Second, the acoustic modulation (generated by the vortex generator) of the incoming flow is started, and the forcing signal (or acoustic frequency) is adjusted such that the corresponding velocity oscillation amplitude, u′, reaches 10% of the mean flow velocity, ū. This fluctuation amplitude has been chosen as a good compromise between significant changes in the flame surface area and a linear response of the flame. The forcing amplitude may be adjusted at each frequency and each operating pressure.

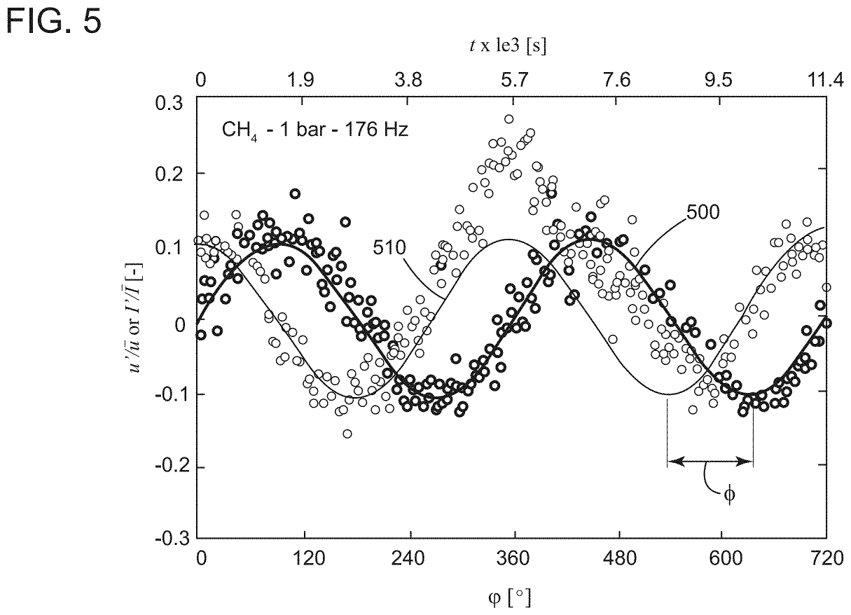

An example of samples (dots) and averaged signals (lines) of the velocity 500 and OH* chemiluminescence 510 is shown in FIG. 5. These measurements have been obtained for the reference flame. The instantaneous values can be significantly different than the average ones. This is mainly due to the turbulent nature of the flow.

FIG. 5 show the temporal evolution of the velocity u′ (curve 500) of the fuel through the fuel testing device and intensity I′ (curve 510) or heat of the flame over 10 s. One will note that the two curves are not in phase due to the phase mismatch Φ. The value of the phase mismatch Φ (which can be measured from FIG. 5) is used later for indicating the increased or decreased potential for thermoacoustic instabilities.

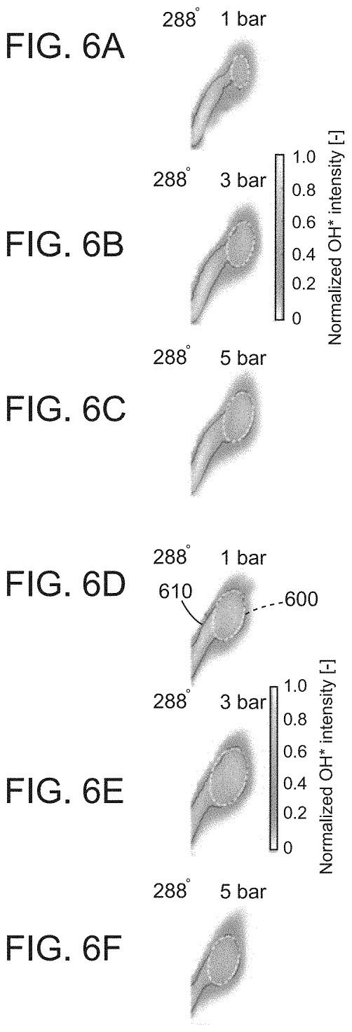

Using the visualization system 150, images for the flames are acquired. For example, methane flames at various pressures are acquired (see FIGS. 6A to 6C) or propane flames at various pressures are acquired (see FIGS. 6D to 6F) for an acoustic forcing at 176 Hz and a phase of 288°. The flame ball is then determined by considering a perimeter of the largest ellipse 600 that can fit into the contour 610 of the top of the flame, at any phase of the forcing period, as illustrated in FIGS. 6A to 6F. In this way, the maximal size of the flame ball has been measured for step 310.

Continuing to the method in FIG. 3B, in step 312, the process compares the maximal size 600 of the flame ball obtained for the test fuel with the maximal size of the flame ball obtained for a reference fuel under the same conditions. If the flame ball of the test fuel is smaller than that of the reference fuel, then the method concludes in step 314 that the test fuel is less sensitive to vortex perturbations than the reference fuel. If the flame ball of the test fuel is larger than that of the reference fuel, the method concludes in step 316 that the test fuel is more sensitive to vortex perturbations than the reference fuel.

The method does not have to stop here. The method may advance to step 318 to also compare the length L of the flame for the test fuel to that of the reference fuel with the same reactant bulk velocity and without operating the vortex generator. This test determines the turbulent burning velocity of the flame and allows inferring the phase difference Φ between the acoustic perturbation (velocity u in FIG. 5) and the flame heat release fluctuation (intensity I in FIG. 5) due to the vortex roll-up when the vortex generator is activated. If the acoustic perturbation and the heat release fluctuation are in phase, then the method concludes in step 320 that there is an increased potential for thermo-acoustic instabilities. If the velocity perturbation and the heat release fluctuation are out of phase, the method concludes in step 322 that there is a decreased potential for thermoacoustic instabilities.

Based on the information obtained in steps 314, 316, 320, and 322, the method assesses in step 324 the propensity of the new fuel to generate thermo-acoustics instabilities. In comparison to the reference fuel, if the test fuel is more sensitive to vortex perturbations (output of step 316) and the phase difference between the acoustic perturbation and the flame heat release fluctuation is reduced (output of step 320), then the test fuel will burn with more propensity to generate thermoacoustic instabilities than the reference fuel. If the test fuel is less sensitive to vortex perturbation (output of step 314) and the phase difference between the acoustic perturbation and the flame heat release fluctuation is increased (output of step 322), then the test fuel will burn with less propensity to generate thermoacoustic instabilities than the reference fuel and in this case the new fuel can safely be used in the gas turbine.

The method and fuel testing device discussed above advantageously test a new fuel in conditions similar to an actual gas turbine and avoiding any complicated calculations. In addition, the test can be performed in minutes and not hours or days as currently performed. In this respect, the most common way to test a new fuel is to perform full-scale tests in the gas turbine. This is not only time consuming, as noted above, but also expensive and disruptive because the gas turbine has to be taken off. An alternate traditional solution is to measure the flame transfer function (FTF) of a canonical flame burning the test fuel and compare with the FTF of the reference fuel. The FTF characterizes the response of the flame to acoustic fluctuations of the unburned gases over a range of frequencies from a few hertz up to 2 kHz. This approach is time consuming and the analysis is sometimes difficult to do, until the test fuel can be categorized more or less sensitive to the thermoacoustic problem.

A fuel testing device and a method of using the fuel testing device are now discussed. The fuel testing device may include, at a minimum, a combustion chamber 140 having an optical access port 142, a visualization system 150 that acquires images of a flame inside the combustion chamber 140, the images being acquired through the access port 142, and a vortex generator 124 that perturbs a flow of a fuel inside the combustion chamber 140. The images are used to determine a propensity of the fuel to thermoacoustic instabilities. In this embodiment, the combustion chamber 140 has a length less than 2 m, which indicates that the fuel testing device is small and/or portable relative to the gas turbine.

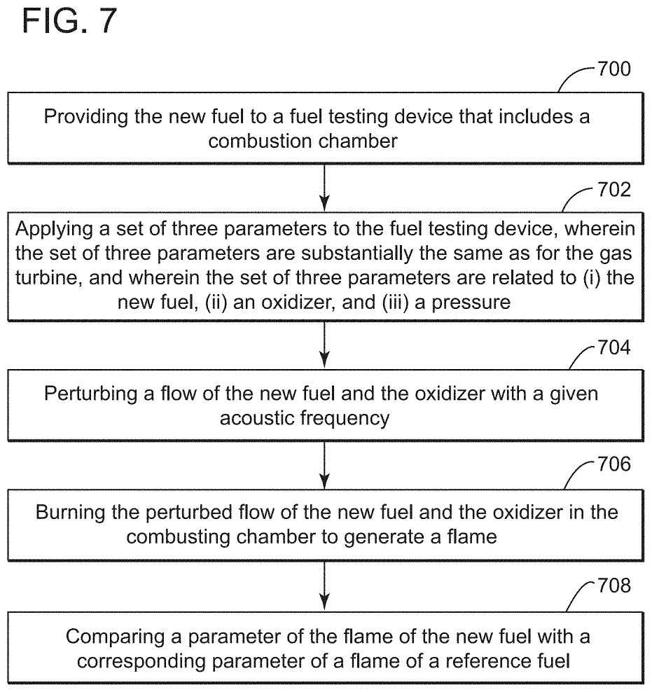

A method for testing with the fuel testing device noted above a new fuel for a gas turbine is now discussed with regard to FIG. 7. The method includes a step 700 of providing the new fuel to a fuel testing device that includes a combustion chamber, a step 702 of applying a set of three parameters to the fuel testing device, wherein the set of three parameters are substantially the same as for the gas turbine, and wherein the set of three parameters are related to (i) the new fuel, (ii) an oxidizer, and (iii) a pressure, a step 704 of perturbing a flow of the new fuel and the oxidizer with a given acoustic frequency, a step 706 of burning the perturbed flow of the new fuel and the oxidizer in the combusting chamber to generate a flame, and a step 708 of comparing a parameter of the flame of the new fuel with a corresponding parameter of a flame of a reference fuel. Based on a result of the comparing step, the new fuel is determined to have more or less thermoacoustic instabilities than the reference fuel.

The set of three parameters includes an equivalence ratio of the new fuel and the oxidizer, a type of the oxidizer, and a pressure inside the combustion chamber. The given acoustic frequency may be between 10 Hz and 1 kHz.

The method may optionally include a step of taking images of the flame inside the combustion chamber with a visualization system, where the visualization system has access to the inside of the combustion chamber through an optical access port. The method may further include a step of calculating with the control and image analysis unit a maximal size of a ball of the flame from the images and/or comparing the maximal size of the ball of the flame of the new fuel with a maximal size of a ball of the flame of the reference fuel burned in the same fuel testing device; and/or estimating a propensity for thermoacoustic instabilities of the new fuel relative to the reference fuel based on a result of the comparing.

The method may also include a step of calculating with the control and image analysis unit a length L of the flame based on the images, and/or, based on the length of the flame of the new fuel and a length of the flame of a reference fuel burned in the same fuel testing device, calculating a phase difference between (1) an acoustic perturbance propagating through the new fuel, and (2) a flame heat release fluctuation due to the given acoustic perturbation; and/or estimating a propensity for thermoacoustic instabilities of the new fuel relative to the reference fuel based on the phase difference.

In one application, the method may include a step of estimating a propensity for thermoacoustic instabilities of the new fuel relative to the reference fuel based on a maximal size of a ball of the flame and a phase difference between (1) an acoustic perturbance propagating through the new fuel, and (2) a flame heat release fluctuation due to the given acoustic perturbation. Further, the method may include setting a value of a ratio of (1) a bulk velocity of the new fuel and (2) a length of the flame.

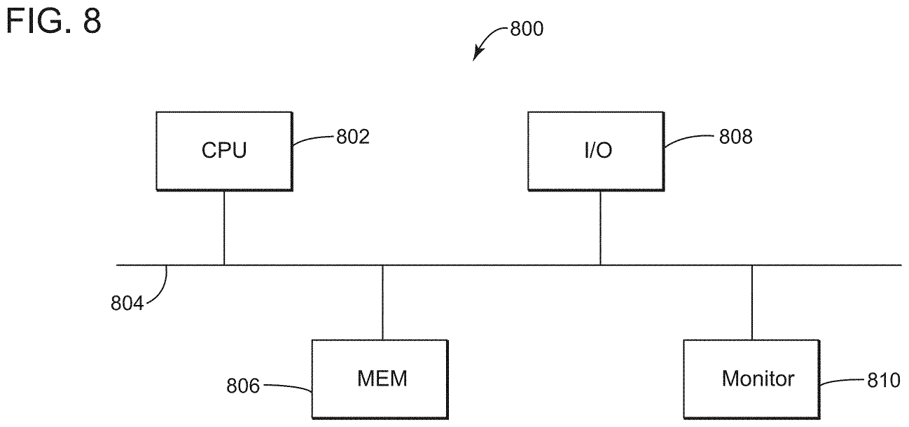

The control and image analysis unit may be implemented in a computing device. The computing device is illustrated in FIG. 8. The computing device 800 includes a processor 802 that is connected through a bus 804 to a storage device 806. Computing device 800 may also include an input/output interface 808 through which data can be exchanged with the processor and/or storage device. For example, a keyboard, mouse or other device may be connected to the input/output interface 808 to send commands to the processor and/or to collect data stored in storage device or to provide data necessary to the processor. In one application, the processor calculates the length of the flame or the maximal size of the ball of the flame, which information may be provided through the input/output interface. Results of this or another algorithm may be visualized on a screen 810.

The disclosed embodiments provide methods and devices that test a fuel propensity to thermoacoustic instabilities. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A fuel testing device comprising:
a combustion chamber having an optical access port;
a visualization system that acquires images of a flame inside the combustion chamber, the images being acquired through the optical access port; and
a vortex generator that perturbs a flow of a fuel inside the combustion chamber,
wherein the images are used to determine a propensity of the fuel to thermoacoustic instabilities, and
wherein the combustion chamber has a length less than 2 m.

2. The device of claim 1, further comprising:
a pressure control system that controls a pressure inside the combustion chamber.

3. The device of claim 2, further comprising:
a control and image analysis unit that analyzes the images and calculates a length L of the flame.

4. The device of claim 3, wherein the control and image analysis unit further calculates a maximal size of a ball of the flame.

5. The device of claim 4, wherein the control and image analysis determines, based on the length L of the flame and the maximal size of the ball of the flame, the propensity of the fuel to thermoacoustic instabilities.

6. The device of claim 1, wherein the fuel testing device is portable.

7. The device of claim 1, wherein an equivalence ratio of the fuel to an oxidizer, the oxidizer, and a pressure inside the combustion chamber are selected to be substantially the same with those in an actual gas turbine.

8. A method for testing a new fuel for a gas turbine, the method comprising:
providing the new fuel to a fuel testing device that includes a combustion chamber;
applying a set of three parameters to the fuel testing device, wherein the set of three parameters are substantially the same as for the gas turbine, and wherein the set of three parameters are related to (i) the new fuel, (ii) an oxidizer, and (iii) a pressure inside the combustion chamber;
perturbing a flow of the new fuel and the oxidizer with a given acoustic frequency;
burning the perturbed flow of the new fuel and the oxidizer in the combustion chamber to generate a flame; and
comparing a parameter of the flame of the new fuel with a corresponding parameter of a flame of a reference fuel,
wherein, based on a result of the comparing step, the new fuel is determined to have more or less thermoacoustic instabilities than the reference fuel.

9. The method of claim 8, wherein the set of three parameters includes an equivalence ratio of the new fuel and the oxidizer, a type of the oxidizer, and a pressure inside the combustion chamber.

10. The method of claim 8, wherein the given acoustic frequency is between 10 Hz and 1 kHz.

11. The method of claim 8, further comprising:
taking images of the flame inside the combustion chamber with a visualization system,
wherein the visualization system has access to the inside of the combustion chamber through an optical access port.

12. The method of claim 11, further comprising:
calculating with the control and image analysis unit a maximal size of a ball of the flame from the images.

13. The method of claim 12, further comprising:
comparing the maximal size of the ball of the flame of the new fuel with a maximal size of a ball of the flame of the reference fuel burned in the same fuel testing device; and
estimating a propensity for thermoacoustic instabilities of the new fuel relative to the reference fuel based on a result of the comparing.

14. The method of claim 11, further comprising:
calculating with the control and image analysis unit a length L of the flame based on the images.

15. The method of claim 14, further comprising:
based on the length of the flame of the new fuel and a length of the flame of a reference fuel burned in the same fuel testing device, calculating a phase difference between (1) an acoustic perturbance propagating through the new fuel, and (2) a flame heat release fluctuation due to the given acoustic perturbation; and
estimating a propensity for thermoacoustic instabilities of the new fuel relative to the reference fuel based on the phase difference.

16. The method of claim 8, further comprising:
estimating a propensity for thermoacoustic instabilities of the new fuel relative to the reference fuel based on a maximal size of a ball of the flame and a phase difference between (1) an acoustic perturbance propagating through the new fuel, and (2) a flame heat release fluctuation due to the given acoustic perturbation.

17. The method of claim 8, further comprising:
setting a value of a ratio of (1) a bulk velocity of the new fuel and (2) a length of the flame to be a given constant.

18. A method for testing a new fuel for a gas turbine, the method comprising:
providing the new fuel to a fuel testing device that includes a combustion chamber;
applying a set of three parameters to the fuel testing device, wherein the set of three parameters are substantially the same as for the gas turbine, and wherein the set of three parameters are related to (i) the new fuel, (ii) an oxidizer, and (iii) a pressure;
perturbing a flow of the new fuel and the oxidizer with a given acoustic frequency;
burning the perturbed flow of the new fuel and the oxidizer in the combusting chamber to generate a flame;
comparing a parameter of the flame of the new fuel with a corresponding parameter of a flame of a reference fuel; and
estimating a propensity for thermoacoustic instabilities of the new fuel relative to the reference fuel based on a maximal size of a ball of the flame and a phase difference between (1) an acoustic perturbance propagating through the new fuel, and (2) a flame heat release fluctuation due to the given acoustic perturbation.

19. The method of claim 18, wherein a control and image analysis determines, based on the length of the flame and the maximal size of the ball of the flame, the propensity of the fuel to thermoacoustic instabilities.

20. The method of claim 18, wherein an equivalence ratio of the fuel to an oxidizer, the oxidizer, and a pressure inside combustion chamber are selected to be substantially the same with those in an actual gas turbine.
